(19) United States
(12) Patent Application Publication
(10) Pub. No.: US 2010/0240971 A1
(43) Pub. Date: Sep. 23, 2010

(54) INTEGRATED MULTIMODALITY BRAIN MONITORING DEVICE

(76) Inventor: Paolo Zanatta, Treviso (IT)

Correspondence Address: BRYAN W. BOCKHOP, ESQ.;BOCKHOP & ASSOCIATES, LLC, 2375 MOSSY BRANCH DR., SNELLVILLE, GA, 30078, US

(21) Appl. No.: 12/726,164

(22) Filed: Mar. 17, 2010

Related U.S. Application Data
(60) Provisional application No. 61/160,923, filed on Mar. 17, 2009.

(51) Int. Cl. A61B 5/1455; A61B 5/0476; A61B 8/00; A61B 5/0488

(52) U.S. Cl. 600/323; 600/544; 600/453; 600/546

(57) ABSTRACT

A device for monitoring brain parameters in a patient includes at least one central nervous system function sensor, at least one brain oxygen sensor, at least one blood flow velocity sensor, a video monitor and a computational circuit. The nervous system function sensor is configured to sense a nervous system function of the patient. The brain oxygen sensor is configured to sense a brain oxygen concentration of the patient. The brain blood flow velocity sensor is configured to sense the blood flow velocity of the patient. The computational circuit is in data communication with the nervous system function sensor and the brain oxygen sensor. The computational circuit is configured to generate a graphic representation, for display on the video monitor, of the nervous system function of the patient and the brain oxygen concentration of the patient.

### CROSS-REFERENCE TO RELATED APPLICATION(S)

This application claims the benefit of U.S. Provisional Patent Application Ser. No. 61/160,923, filed Mar. 17, 2009, the entirety of which is hereby incorporated herein by reference.

### BACKGROUND OF THE INVENTION

1. Field of the Invention

The present invention relates to medical monitoring systems and, more specifically, to a system that monitors a plurality of nervous system functions during surgical operations.

2. Description of the Related Art

Cardiac surgery can be stressful to many different physiological systems. While cardio and pulmonary systems are clearly affected by cardiac surgery, other critical systems can be affected as well. For example, neurological injuries can occur as a result of activities occurring during cardiac surgery. Such neural injuries, even if temporary, can have a serious impact on other systems. Therefore, it is important to monitor indicators of neural health during cardiac surgery.

Monitoring such indicators can result in early detecting of brain ischemia, which allows for early treatment. Several parameters that may be monitored to indicate neural physiology include: Evoked potentials (EP); Electroencephalogram (EEG); electromyography (EMG); brain blood flow velocity with Trans Cranial Doppler (TCD), and brain oxymetry with Near Infrared Spectroscopy (NIRS); and Oxygen venous jugular saturation (SJO₂).

In current operating room environments, several neural parameters may be measured. However, each such parameter is typically measured by a separate device. Because of the amount of clutter associated with several different devices, each of which has its own output display, clinicians often limit the number of neural parameters monitored during cardiac surgery.

Also, as the data from different devices is typically displayed on separate monitors, it is difficult for the clinician to relate the output of one sensor to the output of another sensor. Coordinating events from different sensors can provide important information in real time regarding the patient's current state.

Therefore, there is a need for an integrated brain monitor device that gives clinicians the ability to detect a brain ischemia and the etiopatogenetic mechanisms that produced brain injury early, thereby giving clinicians the ability to treat them.

There is also a need for a system that provides information to clinicians regarding neural responses to cardiac surgery that can be used to develop safer surgical procedures.

There is also a need for a system that displays neural data on a single monitor so as to be easily interpreted and so as to minimize operating room clutter.

### SUMMARY OF THE INVENTION

The disadvantages of the prior art are overcome by the present invention which, in one aspect, is a device for monitoring brain parameters in a patient that includes at least one nervous system function sensor, at least one brain oxygen sensor, a video monitor and a computational circuit. The central nervous system function sensor is configured to sense a central nervous system function of the patient. The brain oxygen sensor is configured to sense a brain oxygen concentration of the patient. The computational circuit is in data communication with the central nervous system function sensor and the brain oxygen sensor. A stimulator circuit is configured to apply an electrical stimulation to a selected location on the patient. The computational circuit is configured to generate a graphic representation, for display on the video monitor, of the central nervous system function of the patient and the brain oxygen concentration of the patient.

In another aspect, the invention is a monitoring device for monitoring brain parameters in a patient. At least one central nervous system function sensor is configured to sense a central nervous system function of the patient. At least one brain oxygen sensor is configured to sense a brain oxygen concentration of the patient. At least one brain blood flow velocity sensor is configured to sense a brain blood flow velocity of the patient. A stimulator circuit is configured to apply an electrical stimulation to a selected location on the patient. A computational circuit is in data communication with the central nervous system function sensor, the brain blood flow velocity sensor and the brain oxygen sensor. The computational circuit is configured to generate a graphic representation, for display on a video monitor, of the central nervous system function of the patient, the brain blood flow velocity of the patient and the brain oxygen concentration of the patient. The computational circuit includes a memory configured to store central nervous system function data of the patient. A touch screen is juxtaposed on the video monitor. The computational circuit is further configured to receive user inputs from the touch screen indicative of a desired graphic data representation and modify the graphic representation so as to correspond to the desired graphic data representation.

In yet another aspect, the invention is a method of monitoring brain parameters of a patient during a surgical procedure. A first electronic representation of a central nervous system function of the patient is generated using a central nervous system function sensor. A second electronic representation of a brain blood flow velocity of the patient is generated using a brain blood flow velocity sensor. Electrical stimulation may be applied to a selected location of the patient. A third electronic representation of a brain oxygen concentration of the patient generated using a brain oxygen sensor. The first electronic representation, the second electronic representation and the third electronic representation are processed on a digital computer so as to generate a graphic representation of the central nervous system function, the brain blood flow velocity and the brain oxygen concentration. The graphic representation is displayed on a video monitor.

These and other aspects of the invention will become apparent from the following description of the preferred embodiments taken in conjunction with the following drawings. As would be obvious to one skilled in the art, many variations and modifications of the invention may be effected without departing from the spirit and scope of the novel concepts of the disclosure.

### BRIEF DESCRIPTION OF THE FIGURES OF THE DRAWINGS

FIG. 1 is a schematic diagram of one embodiment in use.

FIG. 2A is an elevational view of a computer monitor displaying four modes of data.

FIG. 2B is an elevational view of a computer monitor displaying one mode of data.

### DETAILED DESCRIPTION OF THE INVENTION

A preferred embodiment of the invention is now described in detail. Referring to the drawings, like numbers indicate like parts throughout the views. Unless otherwise specifically indicated in the disclosure that follows, the drawings are not necessarily drawn to scale. As used in the description herein and throughout the claims, the following terms take the meanings explicitly associated herein, unless the context clearly dictates otherwise: the meaning of “a,” “an,” and “the” includes plural reference, the meaning of “in” includes “in” and “on.”

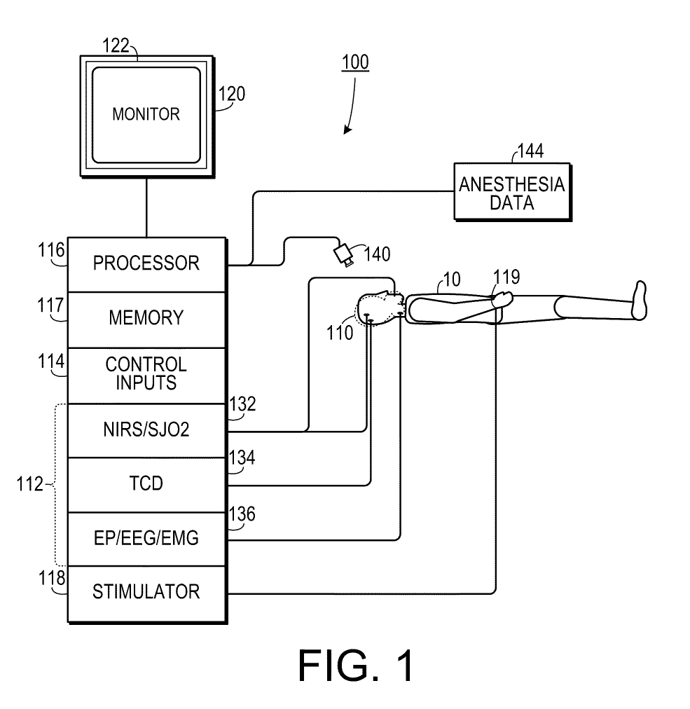

As shown in FIG. 1, one embodiment is device 100 for monitoring brain parameters in a patient 10. The device 110 includes a central nervous system function sensor 136 configured to sense a central nervous system function of the patient. The central nervous system function sensor 136 can include an electroencephalogram (EEG), an evoked potentials (EP) sensor and an electromyogram (EMG) sensor. When an evoked potentials (EP) sensor is employed, a stimulator circuit 118 applies an electrical stimulation to a selected location 119 on the patient 10 (such as the patient's wrist, ankle, head and other locations). When a motor evoked potentials sensor is employed, the other types of evoked potential sensors (i.e., somatosensory, acoustic and visual) are typically not employed.

A brain oxygen sensor 132 senses a brain oxygen concentration of the patient 10. The brain oxygen sensor could include a selected one of a near infrared spectroscopy (NIRS) sensor or an oxygen venous jugular saturation (SJO2) sensor. In some embodiments, a brain blood flow velocity sensor 134 senses a brain blood flow velocity of the patient 10. The blood flow velocity sensor 134 can include a transcranial Doppler (TCD) sensor.

A computational circuit, such as a digital processor 116, is in data communication with the central nervous system function sensor 136 and the brain oxygen sensor 132. The processor 116 generates a graphic representation of the central nervous system function of the patient 10 and the brain oxygen concentration of the patient 10. When brain blood flow velocity sensor 134 is used, the processor 116 also generates a graphic representation of brain blood flow velocity of the patient 10. The processor 116 may also receive anesthesia data from an anesthesia device 144 and display the anesthesia data on the video monitor 120. A memory 117 stores the central nervous system function data of the patient 10 (and, in some embodiments, video data from a video camera 140) for later analysis.

The video camera 140 may be provided to generate a real time surgical view video of the surgical procedure. In this case, the processor 116 also generates an image received from the video camera 140.

A video monitor 120 displays the graphic representation to the clinician and a touch screen 122 is juxtaposed on the video monitor 120 to receive inputs from the clinician. The processor 116 receives user inputs from the touch screen 122 that indicate a desired graphic data representation and the processor 116 modifies the graphic representation so as to correspond to the desired graphic data representation, using well known touch screen and data display software. For example, the clinician might desire one of the data streams regarding the patient's neural function, brain oxymetry and blood flow velocity to be enlarged. To do this, the clinician will touch the touch screen to enlarge the desired function.

One representative embodiment includes a system 100 for monitoring neural physiology in an operating room environment. A plurality of neural physiological indicating sensor elements 110 are applied to the patient 10. These sensors 110 can, for example, can include such sensors as:

Evoked potentials (EP)

Electroencephalogram (EEG)

Electromyography (EMG)

Trans Cranial Doppler (TCD)

Near Infrared Spectroscopy (NIRS)

Oxygen venous jugular saturation (SJO2)

The sensors 110 provide information to a corresponding plurality of sensor electronics modules 112 that perform any necessary signal processing functions on the signals received from the sensors 110. A plurality of control inputs 114 may also be employed to allow the clinician to manipulate the display of data and to control the sensor electronics modules 112. A processor 116 receives data from the sensor electronics modules 112 and the control inputs 114 and processes the data in a format that best serves the needs of the clinician at any given time. The data is displayed on a monitor 120.

A stimulator 118 is used to stimulate the patient 10 to allow acquisition of evoked potentials (including, somatosensory, acoustic, visual and motor). An electrical cable from the stimulator is typically applied to the patient's wrist and ankle for the somatosensory evoked potentials. An electrical cable from the stimulator is typically applied to the patient's eyes and ears for visual evoked potentials (VEP) and the acoustic evoked potentials (BAEP). Application of the cable to the patient's skull may also be employed.

The processor 116 can also record data onto a computer-readable storage medium for post-operation analysis. A digital video camera (not shown) may also be coupled to the processor 116 and the processor 116 can correlate different actions of a clinician with the data received from the plurality of sensors 110. This can also employ data relative to actions performed in administering anesthesia.

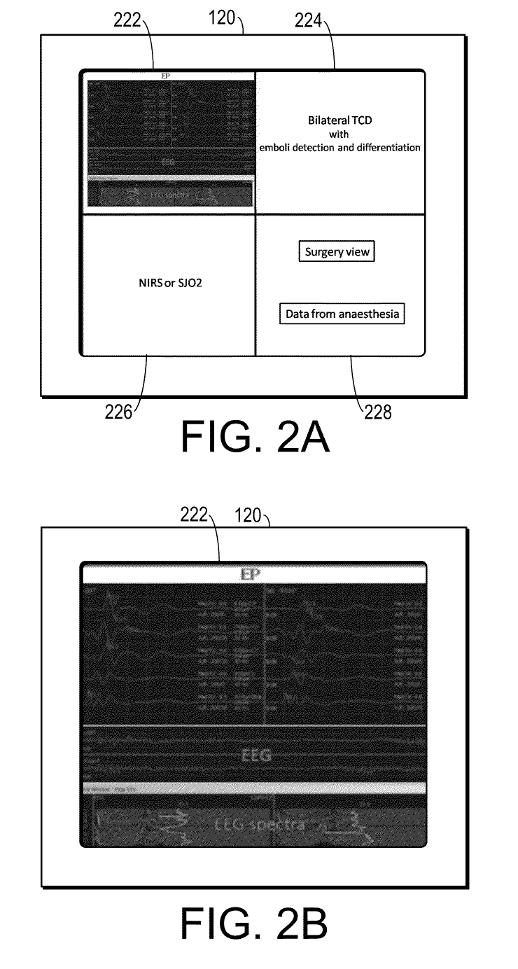

As shown in FIGS. 2A and 2B, the display 120 can be configured to display the data from the sensors the sensors selected by the clinician. For example, as shown in FIG. 2A, the clinician can select to split the screen to display data from four sensors. In this example, evoked potentials data and electroencephalogram data are shown in a first block 222. TCD data with emboli detection and differentiation are shown in a second block 224. NIRS/SJO2 data are shown in a third block 226 and either a surgery view video or anesthesia data (or both) are shown in a fourth block 228. (For the sake of simplicity, the second, third and fourth blocks are labeled and are not graphically represented. In an actual embodiment, these blocks would include actual graphic representations of the corresponding data.)

As shown in FIG. 2B, the clinician can select to have the data from a single sensor (or group of sensors) to be expanded, or even to cover the entire display. In this example, the clinician has expanded the first block 222. The clinician can change the configuration in real time on an as-need basis.

Brain injury during cardiac surgery may depend on brain embolization, low cerebral perfusion pressure, iper-rewarming (iper-rewarming is a mechanism of brain injury due to a faster rate of warming from a cooling state) and/or inflammation induced by the cardiopulmonary bypass. The current method includes analyzing and integrating up to five different kinds of data to detect neural abnormalities during surgery.

Since EP, EEG and EMG explore the nervous system function (stroke/neurocognitive disorders), TCD explores the brain blood flow velocity, the embolic load and emboli differentiation, and NIRS and SJO2 give information about the brain oxygen delivery and consumption, a more thorough understanding of the current state of the patient's neural system is provided to the clinician. Employing the present method allows clinicians to explore the brain function and the etiopathogenetic mechanism that produce the brain injury. In addition, this invention combines all the machines in one, thereby reducing the cost, the need of personnel and the training necessary to perform the monitoring function.

The invention also includes a method of intra-operative brain monitoring supported by a single neuro-monitoring device to reduce the high rate of neurologic injuries produced by the cardiac surgery and other clinical settings in which there is an high rate of neurological complications.

The brain injury in cardiac surgery depends on brain embolization, low cerebral perfusion pressure, iper-rewarming and on inflammation induced by the cardiopulmonary bypass; hypoperfusion and brain embolization mostly depends on surgical maneuvers like clamping and de-clamping of aorta and or epiaortic vessels.

This method includes analyzing and integrating the neuro-physiological signs as evoked potentials (EP), electroencephalogram (EEG), electromyography (EMG), blood flow velocity with transcranial Doppler (TCD), and brain oxymetry with near infrared spectroscopy (NIRS) or oxygen venous jugular saturation (SJO2) in one single monitoring machine.

The integration of such data is vital because the EP, EEG and EMG explore the brain function, the TCD explores the brain blood flow velocity, the embolic and the emboli differentiation and the NIRS and SJO2 provides information about brain oxygen delivery and consumption. In this way it is possible to explore the brain function and the etiopatogenetic mechanism that produce the brain injury.

This monitoring strategy will be easier to apply with a single monitor device and three recording modules: one for EP and EEG and EMG, one for bilateral TCD and one for cerebral oximetry with NIRS or SJO2. Alternate embodiment could employ several brain monitor devices and more than three recording modules.

In the neurosurgical intensive care setting this device and method will give clinicians a best opportunity to monitor the patient brain injury having in a single monitor device the opportunity to explore the brain function (EP, EEG), to diagnose and to follow the effect of the therapy on cerebral vasospasm with TCD and continuously monitor the brain oxygen saturation with SJO2 or NIRS.

The integration of these physiological data would give, in this clinical scenario, a faster diagnosis and therapy of the brain injury. This monitoring device, which can monitor EP, EEG and EMG, could be used in vascular surgery, neurosurgical intensive care and in high risk patient of cerebral ischemia submitted to major surgery.

Several devices can be used for retrieving data in association with the present invention, including: (1) the INVOS® System, available from Somanetics, which can monitor brain oxygen saturation through cerebral oximetry and NIRS; (2) Eclipse Neurological® workstation, available from Axon Systems, which can monitor EP, EEG and EMG; (3) Doppler-Box™, available from Compumedics, which can monitor neural Doppler parameters; and (4) SJO2, available from Edwards Lifescience, which can monitor venous jugular brain oxygen saturation.

The system is able to monitor all kinds of evoked potentials included the motor evoked potentials and the electromyography. This is useful to monitor spinal cord function in aortic vascular aneurism procedures and certain kinds of cardiovascular interventions in the aortic arch, neurosurgical patients and some neurosurgical intensive care patients.

In certain clinical cases (cardiac surgery, vascular surgery, orthopedic surgery in beach chair position, neuro-intensive care patients, and patients having high preoperative risk of stroke submitted to every kind of surgery) one effective brain monitoring strategy includes sensing EEG, bilateral somatosensory evoked potentials (with nerve stimulation from arms and/or legs), transcranial Doppler and NIRS or SJO2.

In some surgical scenarios in which the brain injury does not depend result in an embolism (such as cardiac surgery with extracorporeal circulation), but only in brain hypoperfusion the transcranial Doppler may be optional. In these cases the clinician typically would use EEG, bilateral somatosensory evoked potentials and brain oxymetry with NIRS or SJO2.

The NIRS sensor detects brain oxymetry of a few samples of the brain cortex of the frontal lobe (is a mixed brain cortical oxymetry: 75% venous and 25% arterial). Frequently, cardiovascular patients or patients with high risk factors for stroke have an impairment of the cerebrovascular autoregulatory capacity or they have intracranial vascular stenosis. In these patients brain oxymetry with NIRS may not reflect the real oxymetry of the entire brain. (One can have a good oxygenation of the frontal lobe and an unknown de-saturation of the other cerebral lobes.) In these patients brain oxymetry should be detected with jugular venous oxygen saturation (SJO2) because the data detected from SJO2 reflects the oxygenation state of the entire brain.

In patients with normal autoregulatory capacity and who do not have intracranial vascular stenosis the NIRS method is the good choice for monitoring brain oxymetry because the data detected from the frontal lobe reflects the oxygenation state of the entire brain. Autoregulatory capacity can be measured before surgery with a transcranial Doppler dynamic test. Intracranial vascular stenosis can be detected with angiovascular MRI or angiovascular brain tomography (an angio CT scan).

The method disclosed for detecting brain oxymetry with NIRS (near infrared spectroscopy of the brain cortical tissue) is different from the method to detect the brain oxymetry with SJO2 (jugular venous oxygen).

The EEG, EP and EMG data may be recorded with a common amplifier (box) having a number of bipolar channels. The number of channels used depends on the particular configuration that the clinician desires to monitor. The electrodes on the patient are plugged into the box amplifier's corresponding channels. Complicates cases with also motor EP and EMG sensing can necessitate using a unit with 32 channels. However, in the majority of the cases, a 16 channel unit is sufficient. For simple monitoring solution an eight channel unit (two channel for EEG and six for EP) can be sufficient. The software running on the processor can create the desired display configuration using the same electrode for several different parameters on the patients.

In one embodiment, pattern recognition software is employed to detect a pattern in the data from the TCD device that is characteristic of microembolic signals. Each time a microembolic signal is detected, a counter is incremented and the value of the counter is averaged over time to provide an indication of the microembolic load being detected.

The above described embodiments, while including the preferred embodiment and the best mode of the invention known to the inventor at the time of filing, are given as illustrative examples only. It will be readily appreciated that many deviations may be made from the specific embodiments disclosed in this specification without departing from the spirit and scope of the invention. Accordingly, the scope of the invention is to be determined by the claims below rather than being limited to the specifically described embodiments above.

What is claimed is:

1. A device for monitoring brain parameters in a patient, comprising:
a. at least one nervous system function sensor configured to sense a nervous system function of the patient;
b. at least one brain oxygen sensor configured to sense a brain oxygen concentration of the patient;
c. a video monitor; and
d. a computational circuit in data communication with the nervous system function sensor and the brain oxygen sensor, the computational circuit configured to generate a graphic representation, for display on the video monitor, of the nervous system function of the patient and the brain oxygen concentration of the patient.

2. The device of claim 1, further comprising at least one brain blood flow velocity sensor configured to sense a brain blood flow velocity of the patient and wherein the computational circuit is in data communication with the brain blood flow velocity sensor and is further configured to generate a graphic representation of brain blood flow velocity of the patient.

3. The device of claim 2, wherein the brain blood flow velocity sensor comprises a transcranial Doppler (TCD) sensor.

4. The device of claim 1, further comprising a video camera in data communication with the computational circuit, the video camera configured to sense a surgical view of the patient during a surgical procedure, wherein the computational circuit is further configured to generate an image received from the video camera.

5. The device of claim 1, further comprising a touch screen juxtaposed on the video monitor and wherein the computational circuit is further configured to:
a. receive user inputs from the touch screen indicative of a desired graphic data representation; and
b. modify the graphic representation so as to correspond to the desired graphic data representation.

6. The device of claim 1, wherein the central nervous system function sensor comprises an electroencephalogram (EEG) sensor, an evoked potentials (EP) sensor, and an electromyogram (EMG) sensor.

7. The device of claim 6, further comprising a stimulator circuit configured to apply an electrical stimulation to a selected location on the patient.

8. The device of claim 6, wherein the evoked potentials (EP) sensor comprises an evoked potentials (EP) sensor selected from a group consisting of: a motor evoked potentials (MEP) sensor, a somatosensory evoked potentials (SEP) sensor, a visual evoked potentials (VEP), an acoustic evoked potentials (BAEP) sensor.

9. The device of claim 1, wherein the brain oxygen sensor comprises a selected one of a near infrared spectroscopy (NIRS) sensor or an oxygen venous jugular saturation (SJO2) sensor.

10. The device of claim 1, wherein the computational circuit is further configured to:
a. receive anesthesia data from an anesthesia device; and
b. display the anesthesia data on the video monitor.

11. The device of claim 1, further comprising a memory configured to store central nervous system function data of the patient.

12. A monitoring device for monitoring brain parameters in a patient, comprising:
a. at least one nervous system function sensor configured to sense a central nervous system function of the patient;
b. at least one brain oxygen sensor configured to sense a brain oxygen concentration of the patient;
c. at least one brain blood flow velocity sensor configured to sense a brain blood flow velocity of the patient;
d. a stimulator circuit configured to apply an electrical stimulation to a selected location on the patient;
e. a video monitor;
f. a computational circuit in data communication with the nervous system function sensor, the brain blood flow velocity sensor and the brain oxygen sensor, the computational circuit configured to generate a graphic representation, for display on the video monitor, of the nervous system function of the patient, the brain blood flow velocity of the patient and the brain oxygen concentration of the patient, the computational circuit including a memory configured to store central nervous system function data of the patient; and
g. a touch screen juxtaposed on the video monitor and wherein the computational circuit is further configured to:
i. receive user inputs from the touch screen indicative of a desired graphic data representation; and
ii. modify the graphic representation so as to correspond to the desired graphic data representation.

13. The monitoring device of claim 12, wherein the brain blood flow velocity sensor comprises a transcranial Doppler (TCD) sensor.

14. The monitoring device of claim 12, further comprising a video camera in data communication with the computational circuit, the video camera configured to sense a surgical view of the patient during a surgical procedure, wherein the computational circuit is further configured to generate an image received from the video camera.

15. The monitoring device of claim 12, wherein the nervous system function sensor comprises an electroencephalogram (EEG) sensor, an evoked potentials (EP) sensor and an electromyogram (EMG) sensor.

16. The monitoring device of claim 12, wherein the brain oxygen sensor comprises a selected one of a near infrared spectroscopy (NIRS) sensor or an oxygen venous jugular saturation (SJO2) sensor.

17. The device of claim 12, wherein the computational circuit is further configured to:
a. receive anesthesia data from an anesthesia device; and
b. display the anesthesia data on the video monitor.

18. A method of monitoring brain parameters of a patient during a surgical procedure, comprising the steps of:
a. generating a first electronic representation of a nervous system function of the patient using a central nervous system function sensor;
b. generating a second electronic representation of a brain blood flow velocity of the patient using a brain blood flow velocity sensor;
c. generating a third electronic representation of a brain oxygen concentration of the patient using a brain oxygen sensor;
d. processing the first electronic representation, the second electronic representation and the third electronic representation on a digital computer so as to generate a graphic representation of the nervous system function, the brain blood flow velocity and the brain oxygen concentration; and
e. displaying the graphic representation on a video monitor.

19. The method of claim 18, wherein the step of generating a first electronic representation includes receiving data from an evoked potentials (EP) sensor, an electroencephalogram (EEG) sensor and an electromyogram (EMG) sensor.

20. The method of claim 19, further comprising the step of stimulating the patient with an electrical potential so as to create an evoked potential.

21. The method of claim 18, wherein the step of generating a second electronic representation includes receiving data from a transcranial Doppler (TCD) sensor.

22. The method of claim 21, further comprising the step of employing an emboli counting and differentiation application running on the digital computer to count and differentiate emboli sensed by the transcranial Doppler (TCD) sensor.

23. The method of claim 18, wherein the step of generating a third electronic representation includes receiving data from a selected one of a near infrared spectroscopy (NIRS) sensor or an oxygen venous jugular saturation (SJO2) sensor.

24. The method of claim 18, further comprising the step of storing central nervous system function data of the patient in a digital memory.

25. The method of claim 18, presenting video of the surgical procedure on the video monitor.

26. The method of claim 18, further comprising the steps of:
a. receiving a user input from a touch screen juxtaposed on the video monitor, the user input indicative of a desired graphic data representation; and
b. modifying the graphic representation so as to correspond to the desired graphic data representation.
